(19) United States
(12) Patent Application Publication
(10) Pub. No.: US 2002/0160936 A1
(43) Pub. Date: Oct. 31, 2002

(54) HCV E2 PROTEIN BINDING AGENTS FOR TREATMENT OF HEPATITIS C VIRUS INFECTION

(76) Inventors: HOWARD J. WORMAN, NEW YORK, NY (US); NAOTO MAMIYA, CHITA-GUN (JP)

Correspondence Address: JOHN P WHITE, COOPER & DUNHAM LLP, 1185 AVENUE OF THE AMERICAS, NEW YORK, NY, 10036

(21) Appl. No.: 09/407,430

(22) Filed: Sep. 29, 1999

(51) Int. Cl. A01N 37/18; C07H 21/04; C07K 1/00; C07K 14/00; C07K 17/00; A61K 38/00

(52) U.S. Cl. 514/2; 530/350; 536/23.5

(57) ABSTRACT

The present invention provides a method of treating or preventing hepatitis C virus infection in a subject which comprises administering an effective amount of an agent to the subject, wherein the agent is capable of inhibiting the attachment of hepatitis C virus onto cells by specifically binding to the hepatitis C virus envelope E2 protein so as to treat or prevent hepatitis C virus infection. The present invention also provides a method of identifying a compound which can inhibit the attachment of hepatitis C virus onto cells and can treat or prevent hepatitis C virus infection in a subject by inhibiting the binding of hepatitis C virus envelope E2 protein to a cellular protein associated with hepatitis C virus attachment onto cells and their entry into cells, comprising (a) incubating said compound, the hepatitis C virus envelope E2 protein or its variant and said cellular protein capable of specifically binding to said hepatitis C virus E2 protein under a suitable reaction conditions, (b) determining the interactions between the hepatitis C virus envelope E2 protein or its variant and said cellular protein in the presence of said compound, and (c) comparing the interactions in step (b) with the interaction between the hepatitis C virus envelope E2 protein or its variant and said cellular protein in the absence of said compound so as to identify a compound which can inhibit the attachment of hepatitis C virus into a cell.

Throughout this application, various publications are referenced by author and date. Full citations for these publications may be found listed numerically at the end of the specification immediately preceding the claims. The disclosures of these publications in their entireties are hereby incorporated by reference into this application in order to more fully describe the state of the art as known to those skilled therein as of the date of the invention described and claimed herein.

### BACKGROUND OF THE INVENTION

Chronic hepatitis C is a major public health problem and one of the leading worldwide causes of chronic liver disease, cirrhosis and hepatocellular carcinoma (1). Approximately 4 million Americans are chronically infected with hepatitis C virus and as many as 25% of them may eventually develop cirrhosis (2). End-stage liver disease from hepatitis C is now the leading indication for orthotopic liver transplantation in the United States. Hepatitis C virus was identified in 1989 and demonstrated to be the major cause of what was then referred to as non-A, non-B hepatitis (3, 4). Despite major advances in diagnosing chronic hepatitis C and screening the blood supply since that time, almost nothing is known about how the virus infects, kills or transforms cells. For this reason, current therapeutic options are limited and new agents have been difficult to develop.

According to a recent National Institutes of Health Consensus Development Conference Panel Statement on the Management of Hepatitis C (5), there is an urgent need for effective antiviral therapeutics capable of inhibiting hepatitis C virus replication and stopping or delaying the progression of liver disease. The Panel also concluded that a major bottleneck to the drug discovery process is the absence of a readily available cell culture system that is fully permissive for viral replication A small animal model of hepatitis C virus infection is also lacking. For these reasons, novel, alternative approaches must be developed to identify targets for the design of therapeutic agents for the treatment of patients with chronic hepatitis C.

The development of specific drugs against hepatitis C virus has been impeded because there is no non-primate animal model of infection and all attempts to culture the virus have failed. Currently, only non-specific antiviral agents have been used to treat patients with chronic hepatitis C. The only currently approved drugs in the United States are preparations of interferon-alpha and ribavirin. The long-term cure rate of subjects treated with interferon-alpha is less than 10%. The use of ribavirin, in combination with interferon-alpha, has shown slightly better long-term cure, however, still in only a minority of subjects.

Hepatitis C virus is a positive single stranded RNA virus and a member of the Flavivaridae family (3, 6-10). Once hepatitis C virus infects cells, the positive, single-stranded RNA genome is translated into a polyprotein of 3010 to 3033 amino acids, depending upon the strain (6-9). The viral RNA is not capped and translation occurs via internal ribosome entry sites (10, 11). The mechanism of translation from uncapped viral RNA therefore differs from that used by virtually all cellular mRNAs which are capped at their 5′ ends.

The hepatitis C virus polyprotein is proteolytically processed by both host cell and viral proteases into several smaller polypeptides (6-9, 12) (FIG. 1). The major structural proteins are a core protein and two envelope proteins (E1 and E2). Four major non-structural proteins called NS2, NS3, NS4, and NS5, are also generated, two of which, NS4 and NS5, are further processed into smaller polypeptides called NS4A, NS4B, NS5A, and NS5B. The non-structural proteins have various enzymatic activities, such as RNA helicase (NS3), protease (NS2, NS3-NS4A complex) and RNA polymerase (NS5B). NS5A has been implicated in determining sensitivity to interferon.

The hepatitis C virus envelope proteins E1 and E2 interact with hepatocyte plasma membrane proteins that likely mediate the entry of the virus into cells. E2 has been shown to bind to the plasma membranes of cultured cells (13). E1 and E2 may form a heteromeric complex (14) and their association may be necessary for virus binding to cells and for their entry into cells. However, cell surface proteins that function as hepatitis C virus receptors or co-receptors by binding to E1 and E2 have not been identified.

### SUMMARY OF THE INVENTION

The present invention provides a method of treating or preventing hepatitis C virus infection in a subject which comprises administering an effective amount of an agent to the subject, wherein the agent is capable of inhibiting the attachment of hepatitis C virus onto cells by specifically binding to the hepatitis C virus envelope E2 protein so as to treat or prevent hepatitis C virus infection.

The present invention also provides a method of identifying a compound which can inhibit the attachment of hepatitis C virus onto cells by inhibiting the binding of hepatitis C virus envelope E2 protein to a cellular protein associated with hepatitis C virus binding to cells and their entry into cells, comprising (a) incubating said compound, the hepatitis C virus envelope E2 protein or its variant and said cellular protein capable of specifically binding to said hepatitis C virus E2 protein under a suitable reaction conditions, (b) determining the interactions between the hepatitis C virus envelope E2 protein or its variant and said cellular protein in the presence of said compound, and (c) comparing the interactions in step (b) with the interaction between the hepatitis C virus envelope E2 protein or its variant and said cellular protein in the absence of said compound so as to identify a compound which can inhibit the attachment of hepatitis C virus onto cells.

The present invention further provides a method for determining whether a compound can be used for treating or preventing hepatitis C virus infection in a subject, wherein said compound inhibits the binding of hepatitis C virus envelope E2 protein to a cellular protein associated with hepatitis C virus attachment onto cells so as to block the attachment of hepatitis C virus into cells, comprising (a) incubating said compound, the hepatitis C virus envelope E2 protein or its variant and said cellular protein capable of specifically binding to said hepatitis C virus E2 protein or its variant under a suitable reaction conditions, (b) determining the interactions between the hepatitis C virus envelope E2 protein or its variant and said cellular protein in the presence of said compound, and (c) comparing the interactions in step (b) with the interaction between the hepatitis C virus envelope E2 protein or its variant and said cellular protein in the absence of said compound so as to identify a compound which can inhibit the attachment of hepatitis C virus onto cells.

### BRIEF DESCRIPTION OF THE FIGURES

FIG. 1. Diagram of the major processed proteins encoded by the hepatitis C virus genome. The 3010-3033 amino acid polyprotein is processed into several smaller polypeptides. Core, E1 and E2 are structural polypeptides. Core protein is the virus nucleocapsid and E1 and E2 are viral envelope proteins. The major non-structural proteins are NS2, NS3, NS4 and NS5. NS4 is further processed into NS4A and NS4B and NS5 into NS5A and NS5B. NS2 and part of NS3 are proteases that process the viral polyprotein. NS3 also has RNA-helicase activity. NS4A is a cofactor for the NS3 protease and NS5B is an RNA-dependent, RNA polymerase. The functions of NS4B and NS5A are less-well understood but NS5A is thought to play a role in determining sensitivity to interferon.

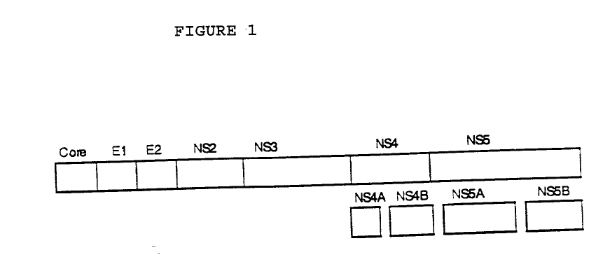

FIG. 2. Amino acid sequence of protein Eo (SEQ ID NO: 1), identified in the yeast two-hybrid assay, that binds to hepatitis C virus envelope protein E2 .

FIG. 3. Binding of Eo to a portion of hepatitis C virus envelope protein E2 (SEQ ID NO: 3) in the yeast two-hybrid assay. The left filter shows yeast colonies co-transformed with a plasmid that encodes a portion of hepatitis C virus E2 fused to the DNA binding domain of GAL4 and a plasmid that encodes a portion of Eo fused to the transcriptional activation domain of GAL4. The colonies give strong β-galactosidase activity (blue; gray in this copy) that is induced by a protein-protein interaction. In contrast, no galactosidase activity is seen in yeast transformed with a portion of Eo alone fused to the transcription activation domain of GAL4 (middle filter) or the Eo fusion protein and a fusion of the DNA binding domain of GAL4 and lamin C (right filter).

FIG. 4. Kyte-Doolittle hydropathy analysis of Eo. The putative transmembrane segment is indicated by *.

FIG. 5. Kyte-Doolittle hydropathy analysis of Eo is shown at top with the putative transmembrane segment indicated by *. Below, the amino acids in Eo, Eo1 and Eo2 are indicated.

FIG. 6. Binding of Eo1 to a portion of hepatitis C virus envelope protein E2 in the yeast two-hybrid assay. The left filter (Eo) shows yeast colonies co-transformed with a plasmid that encodes a portion of Eo fused to the transcriptional activation domain of GAL4. The colonies give strong β-galactosidase activity (blue; gray in this copy) that is induced by a protein-protein interaction. The middle filter (Eo1) shows yeast colonies co-transformed with a plasmid that encodes a portion of hepatitis C virus E2 fused to the DNA binding domain of GAL4 and a plasmid that encodes a portion of hepatitis C virus E2 fused to the DNA binding domain of GAL4 and a plasmid that encodes amino acids 1 to 120 of Eo (Eo1) fused to the transcriptional activation domain of GAL4. The colonies give a weaker β-galactosidase activity (blue; gray in this copy) that is induced by a protein-protein interaction. In contrast, no galactosidase activity is seen in yeast transformed with control plasmids (right filter).

FIG. 7. Amino acid sequence of E2 protein (SEQ ID NO: 2), identified as amino acids 384-746 in FIG. 1 of reference 7.

FIG. 8. Amino acid sequence of a water soluble variant of E2 protein E2 (SEQ ID NO: 3), identified as amino acids 406-660 in FIG. 1 of reference 7.

### DETAILED DESCRIPTION OF THE INVENTION

The present invention provides a method of treating or preventing hepatitis C virus infection in a subject which comprises administering an effective amount of an agent to the subject, wherein the agent is capable of inhibiting the attachment of hepatitis C virus onto cells by specifically binding to the hepatitis C virus envelope E2 protein so as to treat or prevent hepatitis C virus infection.

Variants in amino acid sequence of hepatitis C virus envelope E2 protein are produced when one or more amino acids in naturally occurring hepatitis C virus envelope E2 protein is substituted with a different natural amino acid, an amino acid derivative, a synthetic amino acid, an amino acid analog or a non-native amino acid. Particularly preferred variants include homologous hepatitis C virus envelope E2 protein. Variants of a hepatitis C virus envelope E2 protein may include biologically active fragments of naturally occurring hepatitis C virus envelope E2 protein, wherein sequences of the variant differ from the wild type hepatitis C virus envelope E2 protein sequence by one or more conservative amino acid substitutions. Such substitutions typically would have minimal influence on the secondary structure and hydrophobicity of hepatitis C virus envelope E2 protein.

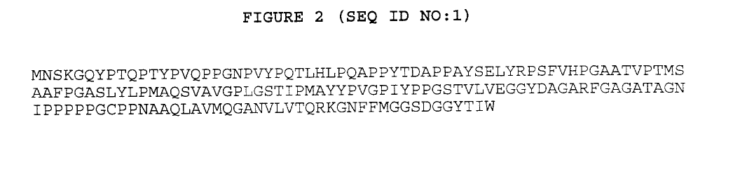

Variants in amino acid sequence of Eo protein are produced when one or more amino acids in naturally occurring Eo protein is substituted with a different natural amino acid, an amino acid derivative, a synthetic amino acid, an amino acid analog or a non-native amino acid. Particularly preferred variants include homologous Eo protein. Variants of an Eo protein may include biologically active fragments of naturally occurring Eo protein, wherein sequences of the variant differ from the wild type Eo protein sequence by one or more conservative amino acid substitutions. Such substitutions typically would have minimal influence on the secondary structure and hydrophobicity of Eo protein. The amino acid sequences of Eo protein has been determined to have SEQ ID NO: 1 in FIG. 2 and the amino acid sequence of one variant of Eo protein has been determined to be amino acids 1-120 of SEQ ID NO: 1.

“Polypeptide” includes both peptides and proteins. “Peptide” means a polypeptide of fewer than 10 amino acid residues in length, and “protein” means a polypeptide of 10 or more amino acid residues in length. In this invention, the polypeptides may be naturally occurring or recombinant (i.e. produced via recombinant DNA technology), and may contain mutations (e.g. point, insertion and deletion mutations) as well as other covalent modifications (e.g. glycosylation and labeling via biotin, streptavidin, fluoracine, and radioisotopes such as ¹³¹I). Moreover, each instant composition may contain more than a single polypeptide, i.e. each may be a monomer (one polypeptide bound to a polymer) or a multimer (two or more polypeptides bound to a polymer or to each other).

As used herein, “effective amount” means an amount of a compound which interrupts the binding between hepatitis C virus E2 protein and a cellular protein, which can inhibit the hepatitis C virus attachment onto cells and can be determined using methods well known to those skilled in the art.

In one embodiment of the method, the agent is a polypeptide, a pseudo enzyme, a peptidomimetic compound, a nucleic acid, an antibody or its variant thereof.

In another embodiment of the method, the cells are liver cells.

In another embodiment of the method, the liver cells are human liver cells.

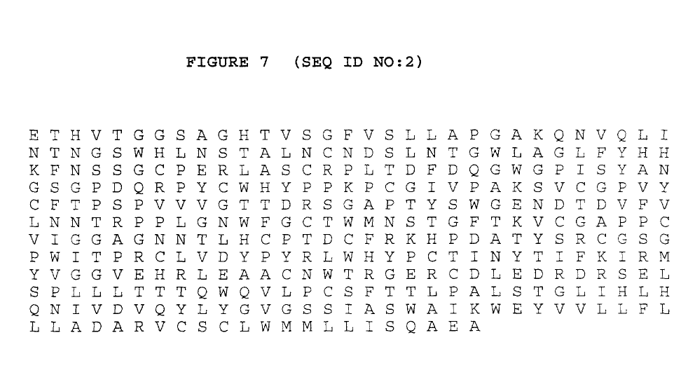

In another embodiment of the method, the hepatitis C virus E2 protein comprises amino acid sequence of FIG. 7, SEQ ID NO: 2.

In another embodiment of the method, the variant of the hepatitis C virus envelope E2 protein comprises 254 amino acids of said hepatitis C virus envelope E2 protein of SEQ ID NO: 2.

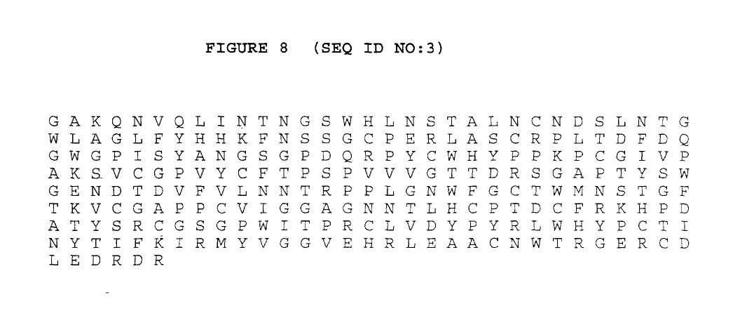

In another embodiment of the method, the variant of the hepatitis C virus envelope E2 protein comprises amino acid sequence of FIG. 8, SEQ ID NO: 3.

In another embodiment of the method, the agent comprises a Eo protein or its variant.

In another embodiment of the method, the Eo protein comprises the amino acid sequence of FIG. 2, SEQ ID NO: 1.

In yet another embodiment of the method, the variant of Eo protein comprises 120 amino acids of SEQ ID NO: 1.

In a further embodiment of the method, the variant of Eo protein comprises Eo1 protein having amino acids 1-120 of SEQ ID NO: 1.

The present invention also provides a method of identifying a compound which can inhibit the attachment of hepatitis C virus into cells by inhibiting the binding of hepatitis C virus envelope E2 protein to a cellular protein associated with hepatitis C virus attachment onto cells and their entry into cells, comprising (a) incubating said compound, the hepatitis C virus envelope E2 protein or its variant and said cellular protein capable of specifically binding to said hepatitis C virus E2 protein or its variant under a suitable reaction conditions, (b) determining the interactions between the hepatitis C virus envelope E2 protein or its variant and said cellular protein in the presence of said compound, and (c) comparing the interactions in step (b) with the interaction between the hepatitis C virus envelope E2 protein or its variant and said cellular protein in the absence of said compound so as to identify a compound which can inhibit the attachment of hepatitis C virus onto a cell.

In one embodiment of the method, the cell is in a subject.

In another embodiment of the method, the subject is a mammal.

In another embodiment of the method, the subject is a human.

In another embodiment of the method, the cells are liver cells.

In another embodiment of the method, the liver cells are human liver cells.

In another embodiment of the method, the cellular protein comprises Eo protein or its variant.

In another embodiment of the method, the Eo protein comprises the amino acid sequence of FIG. 2, SEQ ID NO: 1.

In another embodiment of the method, the variant of Eo protein comprises 120 amino acids of SEQ ID NO: 1. In another embodiment of the method, the variant of Eo protein comprises Eo1 protein having amino acids 1-120 of SEQ ID NO: 1.

In another embodiment of the method, the hepatitis C virus E2 protein comprises amino acid sequence of FIG. 7, SEQ ID NO: 2.

In another embodiment of the method, the variant of the hepatitis C virus envelope E2 protein comprises 254 amino acids of said hepatitis C virus envelope E2 protein of SEQ ID NO: 2.

In another embodiment of the method, the variant of the hepatitis C virus envelope E2 protein comprises amino acid sequence of FIG. 8, SEQ ID NO: 3.

In another embodiment of the method, the inhibition of the attachment of hepatitis C virus onto cells is in vitro.

In yet another embodiment of the method, the compound is not previously known.

In a further embodiment of the method, the previously unknown compound is identified by said method.

The present invention also provides a composition comprising an effective amount of the compound identified by the method which is capable of inhibiting the interactions between hepatitis C virus envelope E2 protein and a cellular protein associated with hepatitis C virus attachment onto cells and their entry into cells.

The present invention also provides a pharmaceutical composition comprising an effective amount of the compound identified by the method which is capable of inhibiting the attachment of hepatitis C virus onto cells.

The present invention further provides a method for determining whether a compound can be used for treating or preventing hepatitis C virus infection in a subject, wherein said compound inhibits the binding of hepatitis C virus envelope E2 protein to a cellular protein associated with hepatitis C virus attachment onto cells and their entry into cells so as to block the attachment of hepatitis C virus into cells, comprising (a) incubating said compound, the hepatitis C virus envelope E2 protein or its variant and said cellular protein capable of specifically binding to said hepatitis C virus E2 protein or its variant under a suitable reaction conditions, (b) determining the interactions between the hepatitis C virus envelope E2 protein or its variant and said cellular protein in the presence of said compound, and (c) comparing the interactions in step (b) with the interaction between the hepatitis C virus envelope E2 protein or its variant and said cellular protein in the absence of said compound so as to identify a compound which can inhibit the attachment of hepatitis C virus onto a cell.

In one embodiment of the method, the subject is a human.

In another embodiment of the method, the compound can be administered orally or by injection.

In another embodiment of the method, the cells are liver cells.

In another embodiment of the method, the liver cells are human liver cells.

In another embodiment of the method, the hepatitis C virus E2 protein comprises amino acid sequence of FIG. 7, SEQ ID NO: 2.

In another embodiment of the method, the variant of the hepatitis C virus envelope E2 protein comprises 254 amino acids of said hepatitis C virus envelope E2 protein of SEQ ID NO: 2.

In another embodiment of the method, the variant of the hepatitis C virus envelope E2 protein comprises amino acid sequence of FIG. 8, SEQ ID NO: 3.

In another embodiment of the method, the cellular protein comprises Eo protein or its variant.

In another embodiment of the method, the Eo protein comprises the amino acid sequence of FIG. 2, SEQ ID NO: 1.

In another embodiment of the method, the variant of Eo protein comprises 120 amino acids of SEQ ID NO: 1.

In another embodiment of the method, the variant of Eo protein comprises Eo1 protein having amino acids 1-120 of SEQ ID NO: 1.

In yet another embodiment of the method, the compound is not previously known.

In a further embodiment of the method, the previously unknown compound is identified by said method.

As used herein, “subject” means any animal, including, for example, mice, rats, dogs, guinea pigs, ferrets, rabbits, and primates. In the preferred embodiment, the subject is a human. “Cells” mean any cells, including, for example, lung cells, and kidney cells. In the preferred embodiment, the cells are liver cells. In a more preferred embodiment, the cells are human liver cells.

“Attachment” means the state of being firmly attached or bound through chemical or physical interactions or both. “Attachment of hepatitis C virus onto cells” means the hepatitis C virus being firmly attached or bound to the cell surface through the interaction between hepatitis C virus proteins, such as HCV E2 protein, and the HCV receptors located at the surface of the cells.

“Entry of HCV into cells” means the penetration of hepatitis C virus through the cell membrane into the cells from the cell surface.

“HCV replication” means HCV reproduction within the cells.

“Hepatitis C virus infection” comprises the attachment of hepatitis C virus to cell surface, the entry of hepatitis C virus into cells, the replication of hepatitis C virus within the cells, and the death or transformation of the cells.

“Agent” means any biological molecule which specifically bind to hepatitis C virus core protein. In one embodiment, the agent is a cellular protein.

As used herein, “suitable reaction conditions” means conditions under which an agent competitively binds to hepatitis C virus E2 protein or a variant thereof.

This invention will be better understood from the Experimental Details which follow. However, one skilled in the art will readily appreciate that the specific methods and results discussed are merely illustrative of the invention as described more fully in the claims which follow thereafter.

Experimental Details

Yeast Two-Hybrid Screening: The yeast two-hybrid assay was performed using previously described methods (15, 16). Yeast two-hybrid screening was performed using the Matchmaker Two-Hybrid System 2 (Clontech). The library screened was a human liver Matchmaker cDNA (HL4002AB, Clontech).

Preparation of Hepatitis C Virus E2 protein construct for two-hybrid screening: The nucleotide sequence encoding amino acids 406-660, of hepatitis C virus E2 protein was amplified by polymerase chain reaction (17) with pHCV-1 as the template. The template for the polymerase chain reaction was pHCV (12) provided by Chiron Corporation. Full length HCV E2 is coded by amino acids 384-746. We therefore made a construct that would express a truncated form of HCV E2 , excluding the hydrophobic segment. The amplified DNA was cloned into the GAL4 DNA binding domain fusion vector pAS2-1 to yield pAS2-1-HCV-E2.9 and library recombinants in the GAL4 activation domain fusion vector pACT2. Screening of 7×106 recombinants of a human liver cell cDNA library, with E2.9 as bait, in the yeast two-hybrid assay, led to the isolation of 5 positive clones three of which encoded portions of Eo.

To amplify the coding region from amino acid 384 to amino acid 661 of hepatitis C virus E2 , oligonucleotides were synthesized based on the known sequence of pHCV (7, 12). The polymerase chain reaction primers had EcoRI and BamHI sites engineered into their 5′ ends to facilitate cloning. The amplified product was cloned into the EcoRI and BamHI sites of plasmid PAS2-1 (Clontech). The identity of the construct was confirmed by restriction endonuclease mapping and DNA sequencing.

Isolation of positive plasmids from yeast: A patch of His⁺, lacZ⁺yeast was incubated with lyticase (Sigma), followed by phenol:chloroform extraction in the presence of glass beads After ethanol precipitation, E. coli HB101 was transformed with isolated yeast DNA, using electroporator II (Invitrogen). Transformants were plated on M9 minimum media plates. Library plasmids were purified with Wizard Plus Maxipreps DNA Purification system (Promega). Library plasmids were then transformed back into yeast Y190 (Clontech). His⁺, lacZ⁺phenotypes were reconfirmed. Autonomous reporter gene activation was checked by co-transformation of the library plasmid with empty PAS2-1. pLAM50-1 (Clontech), which expresses the coding region of lamin C, was used to check non-specific interactions. Plasmids encoding true positive interactors were sequenced.

DNA sequencing: DNA sequencing was performed at the DNA Sequence Facility of the Columbia University Comprehensive Cancer Center.

Results

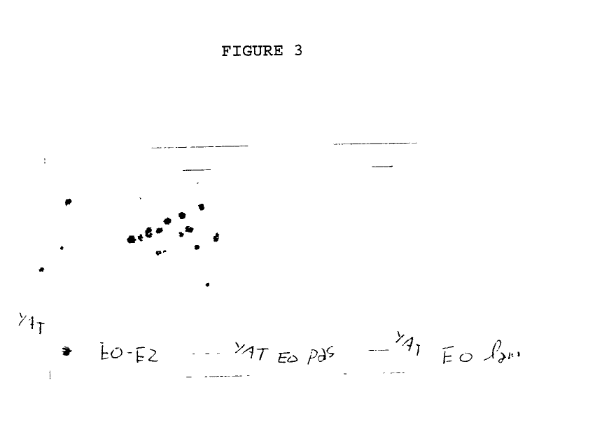

We have screened 7×106 recombinant clones of a hepatocyte cDNA library using the yeast two-hybrid assay (15, 16) with a portion of hepatitis C virus E2 lacking its most hydrophobic, carboxyl-terminal domain. This screening led to the identification of only two true positive clones and both encoded a portion of a protein whose sequence is in the GenBank database (accession number D31767) but whose function is not known. The sequence of this portion, which we call Eo, is shown in FIG. 2. The specificity of binding between Eo and a portion of hepatitis C virus envelope protein E2 in the yeast two-hybrid assay is shown in FIG. 3.

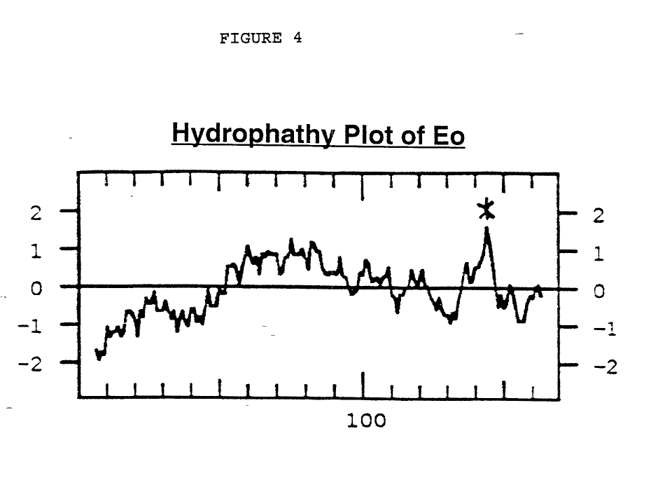

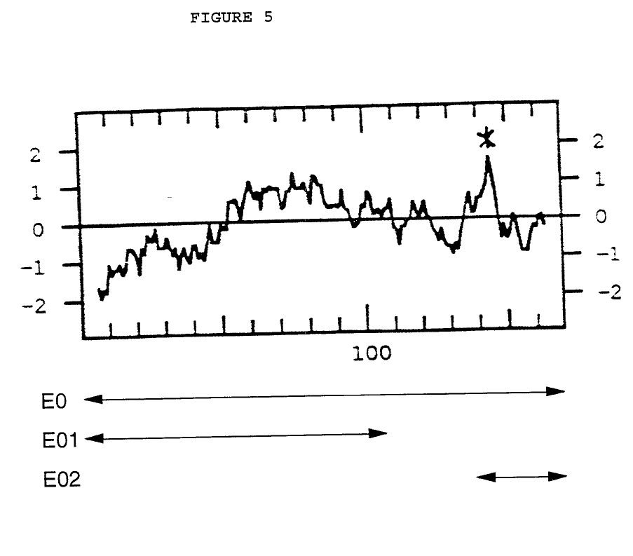

Hydropathy analysis of the amino-acid sequence of Eo shows that it has a hydrophobic stretch that may be a putative transmembrane segment or membrane association domain (FIG. 4). This suggests that Eo may be an integral membrane protein, which would be suspected for a cell surface virus receptor or co-receptor. To further characterize the binding of Eo to hepatitis C virus E2 , two constructs of Eo were engineered, Eo1 and Eo2. Eo1 codes for amino acids 1-120 of Eo and Eo2 codes for amino acids 121-168 of Eo (FIG. 5).

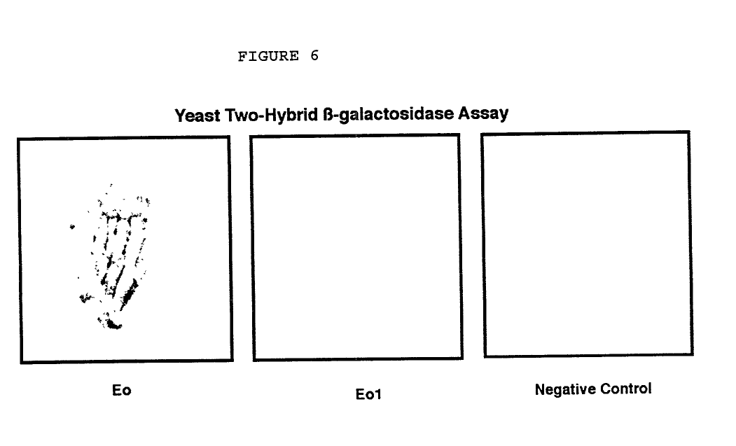

A yeast two-hybrid assay of Y190 cotransformed to express hepatitis C virus-E2 and Eo1 revealed a binding interaction, as did Eo (FIG. 6). Eo2 did not bind to hepatitis C virus E2 . This yeast two-hybrid Eo1-hepatitis C virus E2.9 binding interaction was confirmed in three independent binding assays. Hence, amino acids 1 to 120 of Eo protein binds to hepatitis C virus envelope E2 , however, based on β-galactosidase activity, it appears weaker than the binding of all of Eo protein.

Discussion

The identification of proteins that bind to E1 and/or E2 can potentially function as inhibitors of viral attachment onto cells and their entry into cells, so as to prevent hepatitis C virus infection. This is a new way of preventing hepatitis C virus infection by blocking the HCV attachment onto cells and entry into cells through an external mechanism, i.e. identifying and using compounds that work outside of the cells and that target the envelope proteins E1 and/or E2 proteins. Inhibitors of HCV envelope proteins which bind to critical cellular proteins could also be useful therapeutic agents.

### REFERENCES

1. Mamiya, N., and Worman, H. J. (1997) “Epidemiology, prevention, clinical features and therapy of hepatitis B, C and G.” Curr. Opin. Infect. Dis. 10:3990-397;

2. Alter, M. J. (1997) “Epidemiology of hepatitis C.” Hepatology, 26:62S-65S;

3. Choo, Q. -L., et al. (1989) “Isolation of a cDNA clone derived from blood-borne nonA, nonB viral hepatitis genome” Science, 244:359-362;

4. Kuo, G., et al. (1989) “An assay for circulating antibodies to a major etiological virus of human non-A, non-B hepatitis” Science, 244:359-363;

5. National Institutes of Health Consensus Development Conference Panel Statement (1997) “Management of Hepatitis C” Hepatology, 26:2S-10S;

6. Kato, N., et al. (1990) “Molecular cloning of the human hepatitis C virus genome from Japanese patients with non-A, non-B hepatitis” Proc. Natl. Acad. Sci. U.S.A., 87:9524-9528;

7. Choo, Q. -L., et al. (1991) “Genetic organization and diversity of the hepatitis C virus” Proc. Natl. Acad. Sci. U.S.A., 88:2451-2455;

8. Okamoto, H., et al. (1991) “Nucleotide sequence of the genomic RNA hepatitis C virus isolated from a human carrier: comparison with reported isolates from conserved and divergent regions” J. Gen. Virol., 72:2697-6704;

9. Takamizawa, A., et al. (1991) “Structure and organization of the hepatitis C genome isolated from human carriers” J. Viol., 65:1105-1113;

10. Reynolds, J. E., et al. (1995) “Unique features of internal initiation of hepatitis C virus RNA translation” EMBO J., 14:6010-6020;

11. Fukushi, S., et al. (1997) “The sequence element of the internal ribosome entry site and a 25-kilodalton cellular protein contribute to efficient internal initiation of translation of hepatitis C virus RNA” J. Virol., 71:1662-1666;

12. Selby, M. J., et al. (1993) “Expression, identification and subcellular localization of the proteins encoded by the hepatitis C viral genome” J. Gen. Virol., 74:1103-1113;

13. Rosa, D., et al. (1996) “A quantitative test to estimate neutralizing antibodies to the hepatitis C virus: cytofluorimetric assessment of envelope glycoprotein 2 binding to target cells” Proc. Natl. Acad. Sci. U.S.A., 93:1759-1763;

14. Yi, M., et al. (1997) “Delineation of regions important for heterotrimeric association of hepatitis C virus E1 and E2” Virology, 231:119-129;

15. Fields, S., and Song, O. (1989) “A novel genetic system to detect protein-protein interactions” Nature, 340:245-246;

16. Chien, C., et al. (1991) “The two-hybrid system: a method to identify and clone genes for proteins that interact with a protein of interest” Proc. Natl. Acad. Sci. U.S.A., 88:9578-9582;

17. Saiki, R. K., et al. (1988) “Primer-directed enzymatic amplification of DNA with a thermostable DNA polymerase” Science, 239:487-491

What is claimed is:

1. A method of treating or preventing hepatitis C virus infection in a subject which comprises administering an effective amount of an agent to the subject, wherein the agent is capable of inhibiting the attachment of hepatitis C virus onto cells by specifically binding to the hepatitis C virus envelope E2 protein so as to treat or prevent hepatitis C virus infection.

2. The method of claim 1, wherein the agent is a polypeptide, a pseudo enzyme, a peptidomimetic compound, a nucleic acid, an antibody or its variant thereof.

3. The method of claim 1, wherein the hepatitis C virus envelope E2 protein comprises amino acid sequence of FIG. 7, SEQ ID NO: 2.

4. The method of claim 1, wherein the variant of the hepatitis C virus envelope E2 protein comprises 254 amino acids of SEQ ID NO: 2.

5. The method of claim 1, wherein the variant of the hepatitis C virus envelope E2 protein comprises amino acid sequence of FIG. 8, SEQ ID NO: 3.

6. The method of claim 1, wherein the agent comprises a Eo protein or its variant.

7. The method of claim 6, wherein the Eo protein comprises the amino acid sequence of FIG. 2, SEQ ID NO: 1.

8. The method of claim 7, wherein the variant of Eo protein comprises 120 amino acids of SEQ ID NO: 1.

9. The method of claim 7, wherein the variant of Eo protein comprises Eo1 protein having amino acids 1-120 of SEQ ID NO: 1.

10. The method of claim 1, wherein the cells are liver cells.

11. The method of claim 10, wherein the liver cells are human liver cells.

12. A method of identifying a compound which can inhibit the attachment of hepatitis C virus onto cells by inhibiting the binding of hepatitis C virus envelope E2 protein to a cellular protein associated with hepatitis C virus attachment onto cells and their entry into cells, comprising:
a) incubating said compound, the hepatitis C virus envelope E2 protein or its variant and said cellular protein capable of specifically binding to said hepatitis C virus E2 protein under a suitable reaction conditions,
b) determining the interactions between the hepatitis C virus envelope E2 protein or its variant and said cellular protein in the presence of said compound, and
c) comparing the interactions in step (b) with the interaction between the hepatitis C virus envelope E2 protein or its variant and said cellular protein in the absence of said compound so as to identify a compound which can inhibit the attachment of hepatitis C virus onto cells.

13. The method of claim 12, wherein the cell is in a subject.

14. The method of claim 13, wherein the subject is a mammal.

15. The method of claim 13, wherein the subject is a human.

16. The method of claim 12, wherein the cellular protein comprises Eo protein or its variant.

17. The method of claim 16, wherein the Eo protein comprises the amino acid sequence of FIG. 2, SEQ ID NO: 1.

18. The method of claim 16, wherein the variant of Eo protein comprises 120 amino acids of SEQ ID NO: 1.

19. The method of claim 16, wherein the variant of Eo protein comprises Eo1 having amino acids 1-120 of SEQ ID NO: 1.

20. The method of claim 12, wherein the hepatitis C virus envelope E2 protein comprises amino acid sequence of FIG. 7, SEQ ID NO: 2.

21. The method of claim 12, wherein the variant of hepatitis C virus envelope E2 protein comprises 254 amino acids of SEQ ID NO: 2.

22. The method of claim 12, wherein the variant of hepatitis C virus envelope E2 protein comprises amino acid sequence of FIG. 8, SEQ ID NO: 2.

23. The method of claim 12, wherein the inhibition of the attachment of hepatitis C virus onto cells is in vitro.

24. The method of claim 12, wherein the compound is not previously known.

25. The compound identified by the method of claim 24.

26. A composition comprising an effective amount of the compound identified by the method of claim 12, wherein the compound is capable of inhibiting the interactions between hepatitis C virus envelope E2 protein and a cellular protein associated with hepatitis C virus attachment onto cells and their entry into cells.

27. A pharmaceutical composition comprising an effective amount of the compound identified by the method of claim 12, wherein the compound is capable of inhibiting the attachment of hepatitis C virus onto cells.

28. The method of claim 12, wherein the cells are liver cells.

29. The method of claim 28, wherein the liver cells are human liver cells.

30. A method for determining whether a compound can be used for treating or preventing hepatitis C virus infection in a subject, wherein said compound inhibits the binding of hepatitis C virus envelope E2 protein to a cellular protein associated with hepatitis C virus attachment onto cells and their entry into cells so as to block the attachment of hepatitis C virus onto cells, comprising:
a) incubating said compound, the hepatitis C virus envelope E2 protein or its variant and said cellular protein capable of specifically binding to said hepatitis C virus E2 protein under a suitable reaction conditions,
b) determining the interactions between the hepatitis C virus envelope E2 protein or its variant and said cellular protein in the presence of said compound, and
c) comparing the interactions in step (b) with the interaction between the hepatitis C virus envelope E2 protein or its variant and said cellular protein in the absence of said compound so as to identify a compound which can inhibit the attachment of hepatitis C virus onto cells.

31. The method of claim 30, wherein the subject is a human.

32. The method of claim 30, wherein the compound can be administered orally or by injection.

33. The method of claim 30, wherein the cellular protein comprises Eo protein or its variant.

34. The method of claim 33, wherein the Eo protein comprises the amino acid sequence of FIG. 2, SEQ ID NO: 1.

35. The method of claim 33, wherein the variant of Eo protein comprises 120 amino acids of SEQ ID NO: 1.

36. The method of claim 33, wherein the variant of Eo protein comprises Eo1 protein having amino acids 1-120 of SEQ ID NO: 1.

37. The method of claim 30, wherein the hepatitis C virus envelope E2 protein comprises amino acid sequence of FIG. 7, SEQ ID NO: 2.

38. The method of claim 30, wherein the variant of the hepatitis C virus envelope E2 protein comprises 254 amino acids of FIG. 7, SEQ ID NO: 2.

39. The method of claim 30, wherein the variant of the hepatitis C virus envelope E2 protein comprises amino acid sequence of FIG. 8, SEQ ID NO: 3.

40. The method of claim 30, wherein the compound is not previously known.

41. The compound identified by the method of claim 40.

42. The method of claim 30, wherein the cells are liver cells.

43. The method of claim 2, wherein the liver cells are human liver cells.
